(19) United States
(12) Patent Application Publication
(10) Pub. No.: US 2006/0157068 A1
(43) Pub. Date: Jul. 20, 2006

(54) Your hunger pangs hypnotic imagery

(76) Inventor: David Beaumont Roberts JR., Garner, NC (US)

Correspondence Address: DAVID BEAUMONT ROBERTS JR., 3701 WOODSIDE RD., GARNER, NC, 27529-2647, US

(21) Appl. No.: 11/037,819

(22) Filed: Jan. 18, 2005

(51) Int. Cl. A61B 19/00

(52) U.S. Cl. 128/898

(57) ABSTRACT

This invention is a Quantitative Process and System for reducing body weight and keeping desired weight after loss to lead a more healthful life. It was written with the help of Divine Providence. Its paramount component is Spiritual Hypnotic Medical Imagery in which the dieter swallows exactly 25 lbs. of stainless steel ball bearings into his/her stomach. Under hypnosis this fools the brain into thinking the stomach is full and therefore forestalls hunger pangs. This allows the other two components of the invention, The Roberts Diet to Lose Weight, and The Roberts Weight Maintenance Menu Regimen to be followed without being hungry. This results in a more healthful lifestyle. Millions of overweight and obese Americans will be helped by this System of Dieting!! It is a Medical Breakthrough!!

### BACKGROUND OF INVENTION

“The prevalence of overweight and obesity has increased substantially over the past several decades. The latest Health and Nutrition Examination Survey (NHANES) data indicate 65 percent of U.S. adults aged 20 years and older are overweight with a body mass index (BMI) of 25 or higher, or obese with a BMI of 30 or higher. In addition, 16 percent of children and adolescents ages 6-19 in the United States are overweight.” (Center for Disease Control, Apr. 19, 2005).

The primary object of this invention is to enable overweight and obese America to lose their overweightness and then be able to keep it off following THE ROBERTS WEIGHT MAINTENANCE MENU REGIMEN. The Dieter must recognize that they must stay on this regimen and not go back to the old ways of eating that lead to overweight and obesity. No more fried foods, sweets, and other high-caloric foods!! ‘If it's good, You can't have it’!!!

The most important object of this invention is to forestall Hunger Pangs!! And in some persons it even stops Hunger Pangs, notably the inventor! The mechanism that enables this to happen is Spiritual & Medical Hypnosis, using the Hypnotic Imagery specified in the DETAILED DESCRIPTION OF INVENTION. The paramount idea in the lengthy IMAGERY is the useful and patentable idea that the Dieter, under hypnosis, swallows precisely 25 pounds of stainless steel ball bearings into his stomach, thus fooling the brain into thinking their stomach is full. Therefore the Dieter feels little or no hunger.

The principle reason most diets fail is because they are not satisfying enough and Dieter overeats on fatting and sweet foods to overcome their hunger. This invention in all its utility prevents this from happening! They don't get hungry because of the Spiritual & Medical Hypnotic Imagery, ie, that of swallowing exactly 25 lbs. of stainless steel ball bearings.

Now how can this invention help ‘65 percent+Americans’ achieve healthy body weight? This speaks to the millions of Americans who are overweight, etc. Many diseases are aggravated and worsened by overweight and obesity. It is an unhealthly bodily condition. So this invention is very useful! First of all it will be marketed to Physicians, and the Doctor will have THE ROBERTS DIET TO LOSE WEIGHT and THE ROBERTS WEIGHT MAINTENANCE MENU REGIMEN and The Spiritual & Medical Hypnotic Imagery. The Physician buys the Diets which are copyrighted. Then the inventor and doctor chooses a Hypnotist to read or play a tape of The Spiritual & Medical Hypnotic Imagery. He pays a royalty fee to the inventor for use of the Imagery as specified in this application. This is the epitome of utility to enable millions of Americans to live a healthful and long life!

A paramount object of this invention is the promise the inventor made to OUR LORD that 50% of the profits would go toward financing ‘JESUS HOMES’ for the Homeless of the United States. See http://lifedirect.home.mindspring.com, my Ministry's Web Site under the table of contents: “JESUS' SOLUTION TO HOMELESSNESS”.

### SUMMARY OF INVENTION

“The Quantitative Process and System for Reducing Weight Loss and Keeping Desired Weight after Loss” is a new and revolutionary method for losing body weight and maintaining that loss by The Roberts Weight Maintenance Menu Regimen.

There are three parts to the invention:
1. The Spiritual & Medical Hypnotic Imagery that forestalls Hunger Pangs, etc.
2. The Roberts Diet to Lose Weight Menu.
3. The Roberts Weight Maintenance Menu Regimen.

The principal concept that is new, useful, and not obvious is the idea of swallowing exactly 25 lbs. of stainless steel ball bearings while under hypnosis into the stomach to fool the brain into thinking the stomach is full. This idea is not obvious because it is so new, so useful, and revolutionary that surely it would already be in the marketplace and press!! Because it forestalls Hunger Pangs and allows the dieter to loose weight, it will hinder Bariatric By-Pass Surgery and the millions of dollars spent on over-the-counter diet drugs and prescription diet preparations. And most of all hypnosis is safe! This is a breakthrough in Dieting and keeping overweight weight off!!!

### DETAILED DESCRIPTION OF INVENTION

“Now You are going to go on a Fantastic Journey to a Hypnotic Trance to enable You to lose weight on The Roberts Diet to Lose Weight . . . of about 5 lbs/month . . . perhaps more . . . with exercise.

I want You to relax as much as possible. Are You comfortable in the recliner/sofa? As I am talking to You my voice is going to make You sleepier and drowsier.”

(Hypnotherapist hypnotizes patient to moderate hypnosis and then reads the following or plays a tape of Imagery:)

“Now I want You to imagine in Your Mind's Eye the following: It is fairly dark in front of You, but You are able to make out the entrance of a very long dark tunnel, directly in front of You. This will be the start of Your journey into a Hypnotic Trance that will explain to Your mind and body how You will be able to forestall the pangs of hunger!

Now You have the very long dark tunnel completely ahead of You. In the floor of the tunnel are concrete stepping-stones as well as a handrailing on either side of the tunnel. Now take Your arms and hands and put them on the railings, and step slightly upward on the first concrete stepping-stone. You are going to climb up this upward sloping tunnel. It is dark, but You can easily feel Your way by holding the handrailings and stepping carefully upward on each concrete stepping-stone. Now as You have been listening to my voice, You have become more and more relaxed. You are getting even more drowsier and sleepier as I talk. Now You are slowly climbing upward into this dark tunnel. With every single step You take upward, You are getting more and more sleepier. You are now on the 5th concrete stepping-stone, and every step You have taken You will get more and more sleepier. You have a long way to go, so continue to climb upward. Every step You take You will find You are getting even more sleepier. But You have a firm grip on the handrailings, so You will not fall.

You are now at the 15th concrete stepping-stone, and You have gotten so sleepy you are getting even more drowsy. But by the time You get to the concrete 25th stepping-stone You will be actually sleep-walking. Remember that You are still climbing upward, and the depth of Your sleep is getting so great that your whole body is getting heavier and weightier. Your arms are tired and Your feet are tired. But You can still sleep-walk! Now as You step upward You are also becoming more and more hypnotized. By the 40th stepping-stone You are deeply hypnotized. You are numb all over. The muscles of Your hands, arms, and legs are so relaxed You are numb all over. So, so sleepier and drowsier . . . . While You are in this state of Hypnosis You can still hear my voice and will follow all my instructions. But I want You to continue Your Hypnotic Journey and with every step You take You are getting so, so, so, sleepy!! You are now at the 50th stepping-stone as the depth of Your sleep has gotten so great and so sound! You continue climbing. You are now at the 65th stepping-stone, and You are greatly hypnotized. But incredibly with every step You are take You are more and more soundly asleep.

But now You are beginning to see a glimmer of light that is your destination at the end of the tunnel. You keep climbing and climbing upward, getting more and more hypnotized as You go. Time passes. Now you are seeing a brighter Celestial Light at the end of the tunnel. Remember You are even more and more hypnotized as You climb toward that Celestial Light. You are now at the 75th stepping-stone and You are so, so, so, so, sleepy!! The Celestial Light You are seeing ahead of You is brighter and brighter as You climb upward. And You are getting so sleepy beyond description . . . . You continue to climb. You are now at the 85th stepping-stone. You are getting so hypnotized as You climb upward that when You come into the end of the tunnel and into the brilliance of the Celestial Light You will be close to being in a Hypnotic Trance. The only thing You will hear is my voice, so I will be giving You specific orders that You will not remember when I wake You up, invigorated and feeling renewed.

You are now at the very end of this long dark tunnel you have been climbing as asleep as You are. When You get to the 100th stepping-stone You will stop. You see the wonderful brilliance of the Celestial Light coming from the end of the tunnel . . . . You are so, so, so, so, so, sleepy You are now in a Hypnotic Trance. So now You will heed only my voice as You stare at the brilliant Celestial Light!! Do You see anything?

Look down at Your feet. You will see a bowl of stainless steel ball bearings on the tunnel floor. They are ⅜th inch in diameter. The whole weight of these ball bearings is exactly 25 lbs.

Now I want You to pick up every ball bearing and put it in Your Mouth and swallow it. Don't gulp. (Note to Hypnotherapist: Repeat these two sentences three times). The ball bearings are small enough in diameter so they will easily slide down your esophagus and into Your stomach. This will take You a few minutes to do. (Pause).

The stainless steel ball bearings will not harm Your stomach lining at all and the acid in Your stomach isn't strong enough to corrode your stomach in any way! You will be able to have these ball bearings in Your stomach for many years without any problem. Now this imagery essentially fools your mind into thinking that because of the weight of them (25 lbs.) your stomach is full.

Now the weight of 25 lbs. of weight in your stomach is precise. If you are ever hypnotized by anyone else who has you swallow less than 25 lbs. of ball bearings you are to completely ignore their commands!! Any amount of the weight of ball bearings in your stomach besides the exact weight of 25 lbs. will not forestall hunger pangs and your stomach will not feel full, as if your just eaten a full meal. Is this perfectly clear to you? (Nod your head if you agree!) You will never allow any other hypnotist to hypnotize you and have you swallow less or more than 25 lbs . . . . You will not obey any other hypnotic imagery and you will only have the optimum weight of 25 lbs. lying in your stomach. You can do somersaults and the ball bearings will never fall out!!

Now it is time that You came out of this Hypnotic Trance to help You lose weight.

Now I am going to count from 1 to 15 and You will be able to get out of this Hypnotic Trance. You will remember not a thing of what I have said, and no other Hypnotist will be able to hypnotize you and reveal the secrets of this diet imagery!

Now I am going to arouse You very slowly. I am going to start to count down from 15 slowly, starting right now:
Fifteen: You are in a Hypnotic Trance.
Fourteen: You are starting to come out of Your Hypnotic Trance.
Thirteen: You are now definitely coming out of Your Hypnotic Trance.
Twelve: You are still deeply hypnotized, and are very, very sleepy.
Eleven: You are now becoming less sleepy but still very drowsy.
Ten: You are still hypnotized, but not as sleepy now.
Nine: You are definitely less and less sleepy and drowsy.
Eight: You are still sleepy, but not as much as a minute ago.
Seven: You are now still moderately sleepy and You are just beginning to wake up.
Six: Your sleepiness is wearing off; You are not so drowsy.
Five: You certainly are now waking up gradually.
Four: You have come a long way from Your Hypnotic Trance.
Three: You are positively and absolutely coming out of a deep sleep, and are waking up . . . .
Two: You are just a tiny bit sleepy and are conscious of Your environment.
One: You are now totally aware and feel invigorated and renewed.”

1. This invention is a Quantitative Process and System for reducing weight loss and keeping desired weight after loss.

2. In combination with 1 is a Medical and Spiritual Hypnotic Imagery that is safe and effective for weight loss and comprises swallowing exactly 25 lbs. of stainless steel ball bearings under hypnosis into the stomach to fool the brain into thinking the stomach is full and thereby forestalling hunger pangs. Therefore without hunger, dieters will stay on the Diets, as specified in claim 3.

3. In combination of 1 & 2 is The Roberts Diet to Lose Weight and The Roberts Weight Maintenance Menu Regimen which are both nutritious, safe, effective, and enjoyable to eat.

4. In combination of 1, all The Spiritual & Medical Hypnotic Imagery doesn't have to be reinforced by repeating it again, and will remain effective till death.

5. In combination of 1, 2, & 3 The Roberts Diet to Lose Weight reduces Blood Pressure, LDL, Triglycerides, and Cholesterol levels to more healthy ones.

6. In combination of 1, 2, 3, 4, & 5 this invention will take the place of the hundreds of weight loss remedies and save the overweight and obese American People millions of dollars!
